(19) United States
(12) Patent Application Publication
(10) Pub. No.: US 2010/0003270 A1
(43) Pub. Date: Jan. 7, 2010

(54) WHOLE BLOOD CULTURES COMPRISING STIMULATED IMMUNE CELLS, AND USE THEREOF AS MEDICAMENTS

(76) Inventor: Horst KIEF, Ludwigshafen (DE)

Correspondence Address: FOLEY AND LARDNER LLP;SUITE 500, 3000 K STREET NW, WASHINGTON, DC, 20007, US

(21) Appl. No.: 12/487,886

(22) Filed: Jun. 19, 2009

Related U.S. Application Data
(63) Continuation-in-part of application No. PCT/EP2007/011398, filed on Dec. 21, 2007.

(30) Foreign Application Priority Data
Dec. 22, 2006 (DE) 102006061315.5
May 25, 2007 (DE) 102007024609.0

(51) Int. Cl. A61K 39/00; A01N 1/02; C12Q 1/02

(52) U.S. Cl. 424/184.1; 435/2; 435/29

(57) ABSTRACT

The present invention relates to a whole-blood culture containing specific immunocompetent killer cells that are activated against tumor cells, viruses, bacteria and/or allergens, whereby the whole-blood culture consists of whole-blood and a culture medium at a ratio of 3:1 to 4:1, whereby the culture medium has an oxygen excess of at least 100% or more and contains a water-soluble emulsification product comprising a mixture of phospholipids, vitamin E, and low-molecular proteoglycans with a molecular weight of 1,200 to 12,000 Dalton, and whereby dead tumor cells or fragments thereof and/or viral and/or bacterial antigens and/or allergens have been added to the whole-blood culture [to serve] as antigen in the specific recognition process for production of the activated killer cells (stimulation). The invention also relates to a method for producing the culture, as well as stimulants for the whole-blood culture and a method for the selection thereof.

### CROSS-REFERENCE TO RELATED APPLICATIONS

This application is a continuation-in-part of PCT international application No. PCT/EP/011398 filed on Dec. 21, 2007 and claims the benefit of priority from prior German Patent Application No. 10 2006 061 315.5, filed Dec. 22, 2006 and German Patent Application No. 10 2007 024 609.0, filed May 25, 2007, and the entire contents of each of these applications are incorporated herein by reference.

### BACKGROUND OF THE INVENTION

The present invention relates to whole-blood cultures containing stimulated immunocompetent cells as medicinal agents, and the production thereof.

Since malignant tumors form hematogenic and lymphogenic metastases via single cells and groups of cells that detach from the cell tissue, an autologous blood culture would, in principle, provide an ideal opportunity for culturing killer cells that are specifically activated against tumor cells. The selective killing of tumor cells in a culture of this type can be effected relatively easily by an excess of oxygen in the culture atmosphere. Since the partial or total switch to fermentative metabolism renders tumor cells relatively intolerant to active oxygen species, they are killed earlier as compared to the normal healthy blood cell. These dead or attenuated yet living tumor cells or their fragments then serve as antigen in the specific recognition process of the defense cells developing in a culture of this type. Not only the antigen recognition and processing process requires virtually the entire range of lymphocytes and monocytes, but the interplay of cytokines also requires the matching receptors of a large spectrum of immunocompetent cells, which can be provided only by a whole-blood culture.

Aside from the process of sensitizing immunocompetent cells to tumor antigens in a culture of this type, the innovation also provides the opportunity to specifically select viral and bacterial antigens as well as allergens and use them as immunostimulants.

However, when a culture of this type is supplied with an oxygen excess (10,000 to 30,000 Pascal) in the culture atmosphere, the lifetime of the healthy blood cell, regardless of whether it is red or white, is inevitably limited as well. Vitamin E has proven according to the invention to be an ideal protective measure in this regard. Since alpha-tocopherol, as a solely fat-soluble vitamin (including as the acetate), does not or insufficiently dissolve in a culture medium that is equivalent to an RPM solution (standard medium for cell cultures), it must be admixed to a culture medium of said type as a water-soluble emulsified substance. Phospholipids prove to be ideal emulsifiers due to their physiological likeness to the human cell membrane.

Folic acid as a nucleophilic component can be an essential ingredient of a nutrient medium. In the present case, it surprisingly proves to be an additional emulsifier and thus a very valuable solubilizer for vitamin E. The same is true of low-molecular heparins (molecular weight 1,200 to 12,000 Daltons), but also of other proteoglycans, e.g. pentosan polysulfate, which even has to be conceded to have a cell-proliferative effect according to our experience.

The culture medium (vol: 30-40 ml per 120 ml whole-blood) in this context consists of the typical vitamin-enriched electrolyte and amino acid solution containing 100 mg vitamin E acetate and 10% phospholipids, of which 90% are phosphatidylcholine, 10 mg folic acid, and 100 mg pentosan polysulfate.

### BRIEF DESCRIPTION OF THE DRAWINGS

FIG. 1 shows a graph of the development of the differential blood count in the absence of phospholipids and alpha-tocopherol.

FIG. 2 shows a graph of the development of the differential blood count of cultures grown according to the present method.

FIG. 3 shows a graph of comparison of platelet counts.

FIG. 4 shows a graph of the development of the number of leukocytes in the method described above-subsequent cultures.

FIG. 5 shows a graph of the development of the differential blood count of a subsequent cultures made up of macrophages according to the method described above.

FIG. 6a shows a graph of cumulative plot of immune system-modulating effects of various toxins and antigens on the blood of neurodermatitis patients amount of stainable biomaterial of granulocytes and lymphocytes.

FIG. 6b shows a graph of cumulative plot of immune system-modulating effects of various toxins and antigens on the blood of neurodermatitis patients content of granulocytes and lymphocytes.

FIG. 6c shows a graph of cumulative plot of immune system-modulating effects of various toxins and antigens on the blood of neurodermatitis patients toxicity effects.

FIG. 6d shows a graph of culture transformation assay of cell morphology of granulocytes.

FIG. 6e shows a graph of culture transformation assay of cell morphology and cell count.

FIG. 6f show a graph of cumulative plot of immune system-modulating effects of various toxins on the blood of patients with neurodermatitis.

FIG. 7a shows a graph of culture transformation assay case of multiple sclerosis.

FIG. 7b shows a graph of culture transformation assay of SF3 case of multiple sclerosis.

FIG. 7c shows a graph of culture transformation assay of SF4 case of multiple sclerosis.

FIG. 8 shows a graph of leukocytes stimulation with bacterial ribosomes and phospholipids.

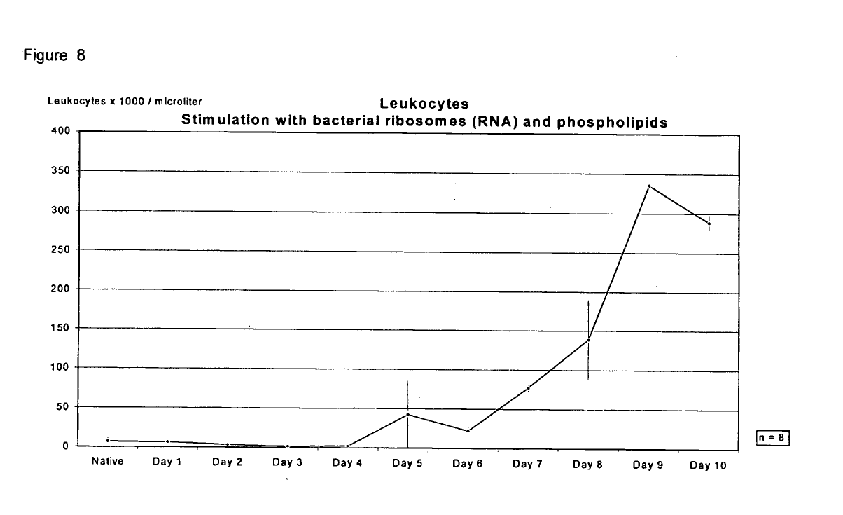

FIG. 9 shows a graph of differential blood count stimulation with bacterial ribosomes and phospholipids.

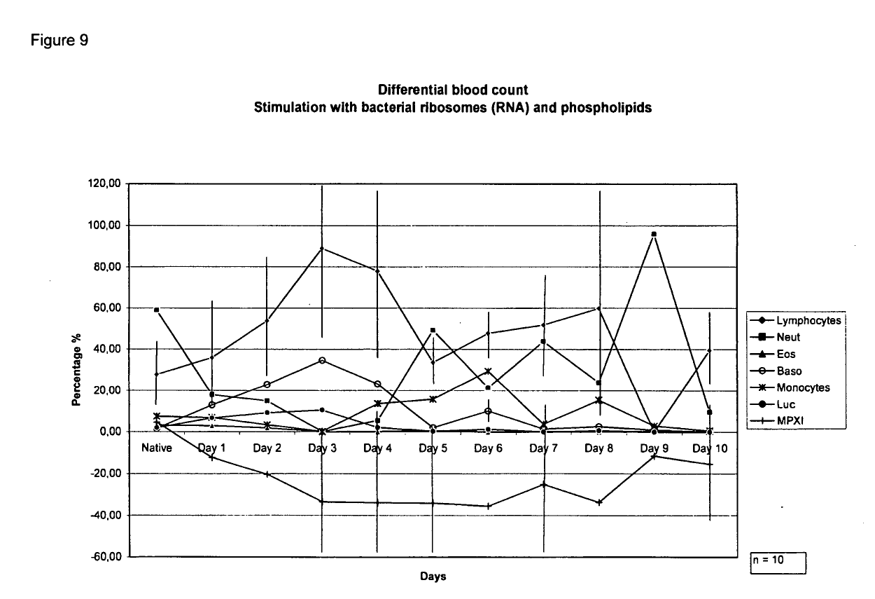

FIG. 10 shows a graph of leukocytes stimulation with influenza virus fraction (hemagglutinin) and phospholipids.

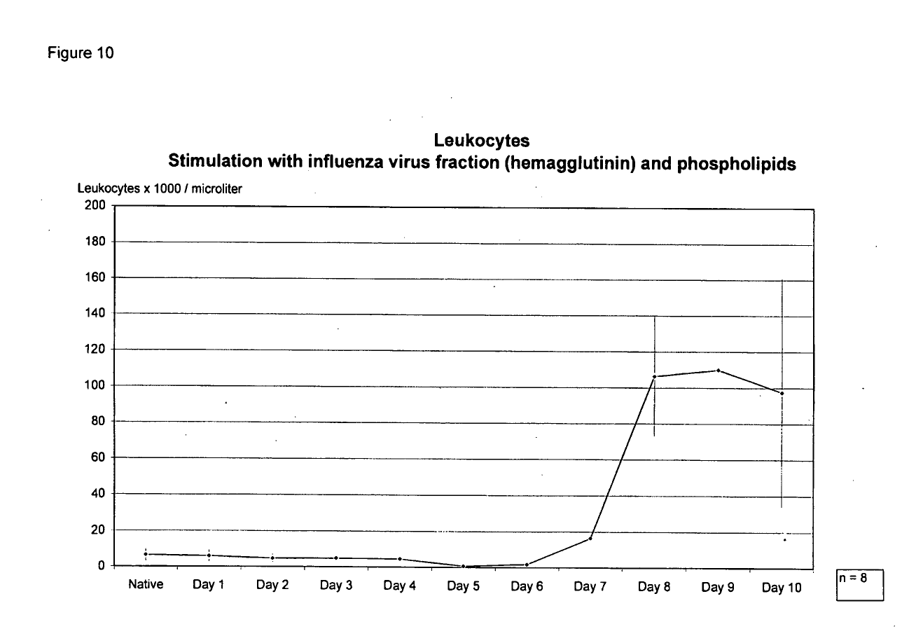

FIG. 11 show a graph of differential blood count stimulation with influenza virus fraction (hemagglutinin) and phospholipids.

FIG. 12 shows a graph of leukocytes stimulation with influenza virus fraction (hemagglutinin), baterial ribosomes and phospholipids.

FIG. 13 shows a graph of differential blood count stimulation with influenza virus fraction (hemagglutinin), baterial ribosomes and phospholipids.

FIG. 14 shows a graph of leukocytes subsequent culture.

FIG. 15 shows a graph of differential blood count subsequent culture.

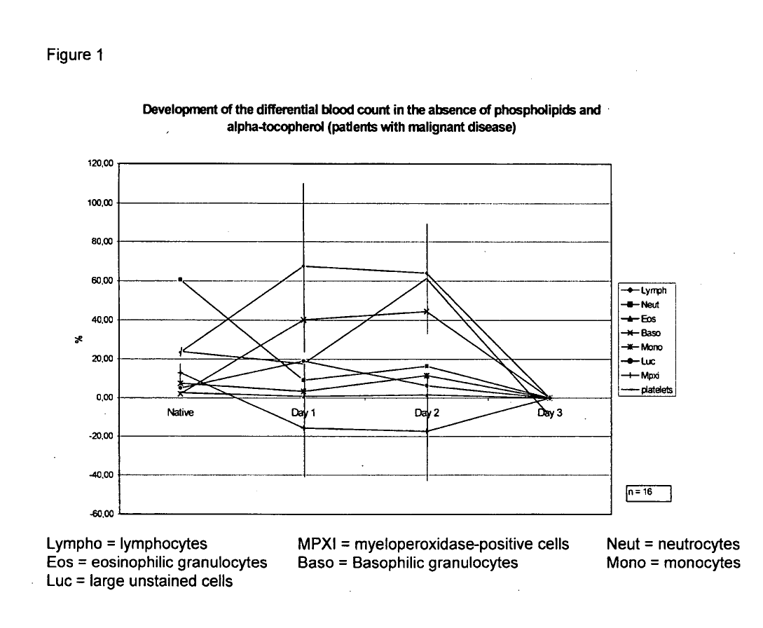

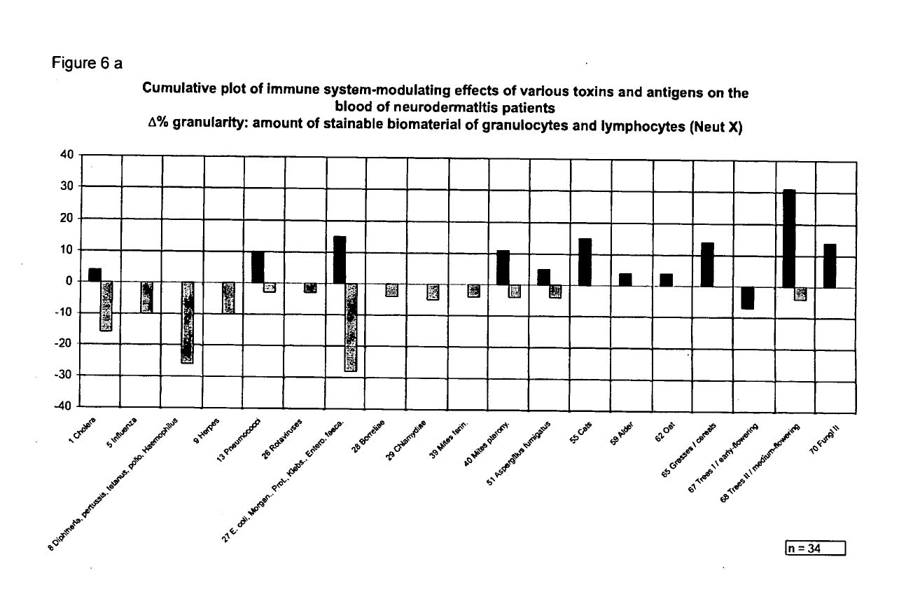

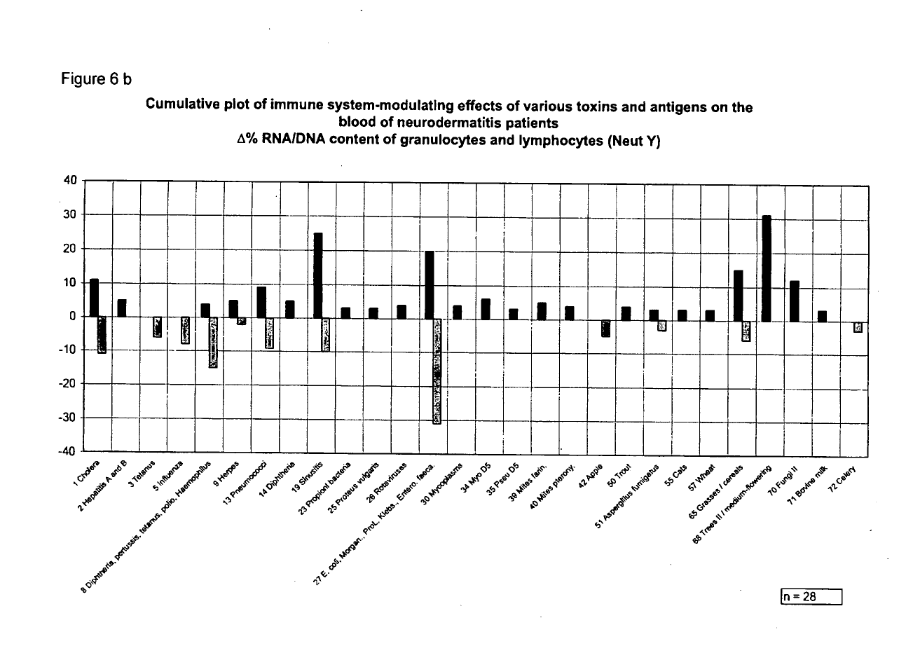

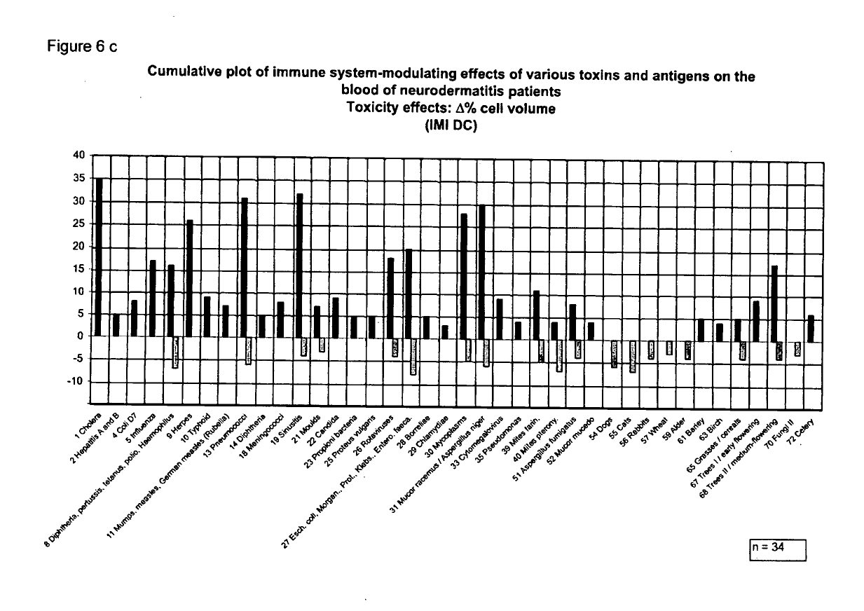

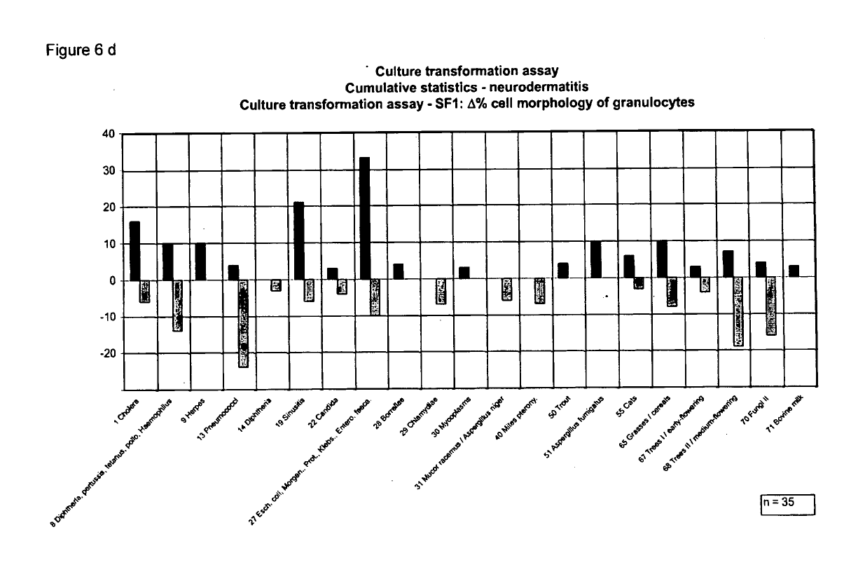

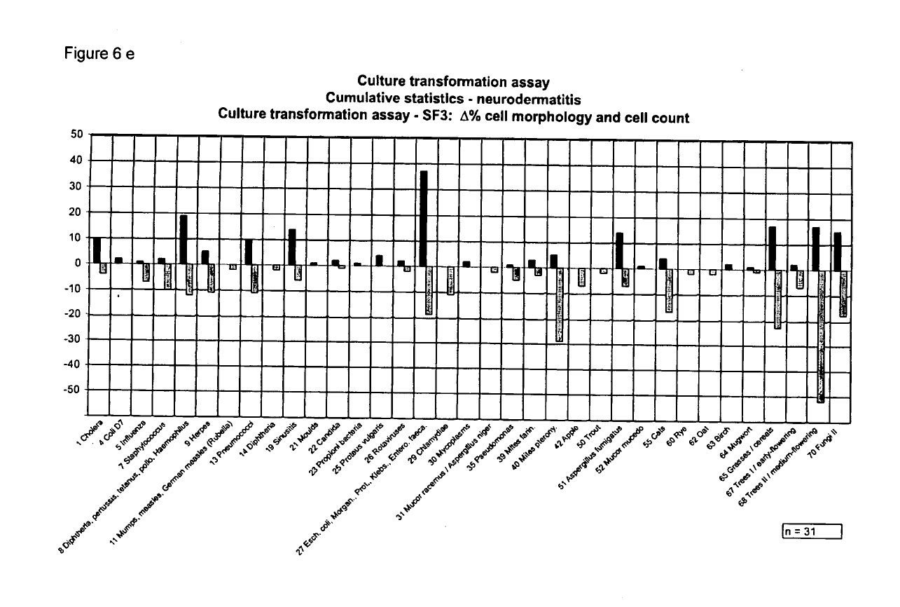

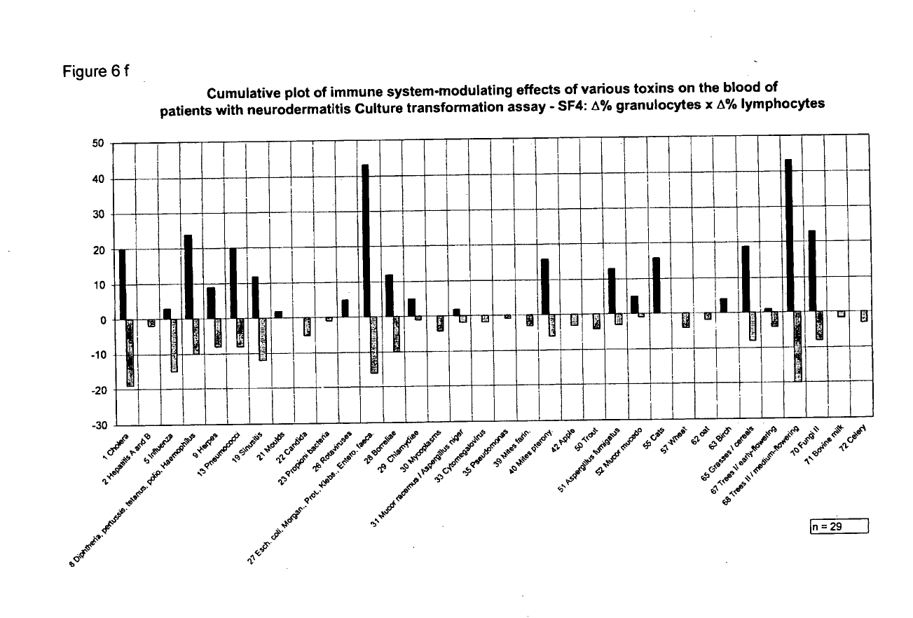

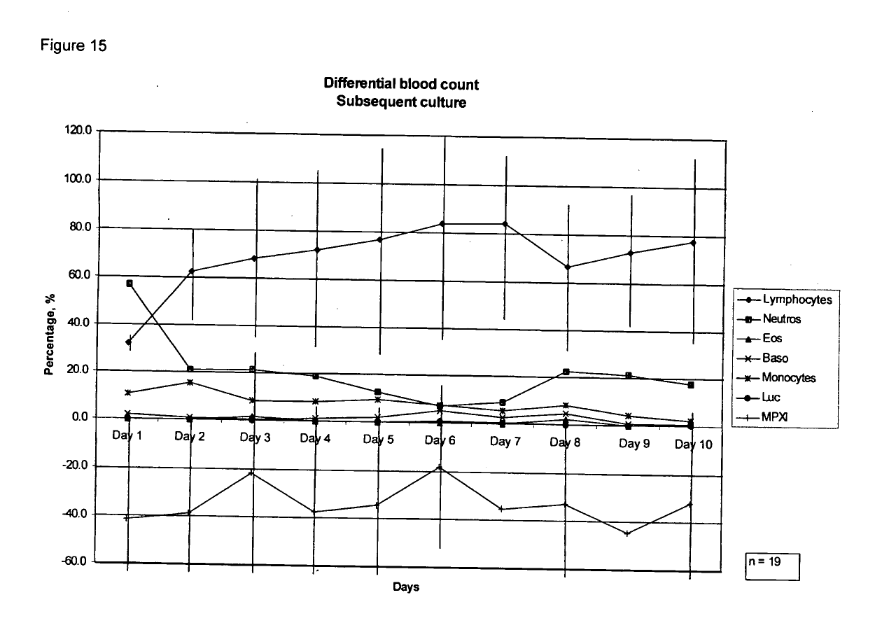

### SUMMARY OF THE INVENTION

Whereas a whole-blood culture survives maximally 3 days in the absence of the supplements specified above (see FIG. 1), a whole-blood culture that contains the supplements specified above proliferates for many days more with an average peak of approx. 100,000 cells (leukocytes) per microliter on day 6 (see FIG. 6).

A whole-blood culture including the erythrocytes is not viable because of the low resistance of the erythrocyte membrane to activated oxygen species. The innovation described herein is advantageous not only in the longer culturing time and ensuing higher yield, but also offers the opportunity of responding to erythrocyte-borne antigens and simplified selection of leukocyte subpopulations as shall be described below.

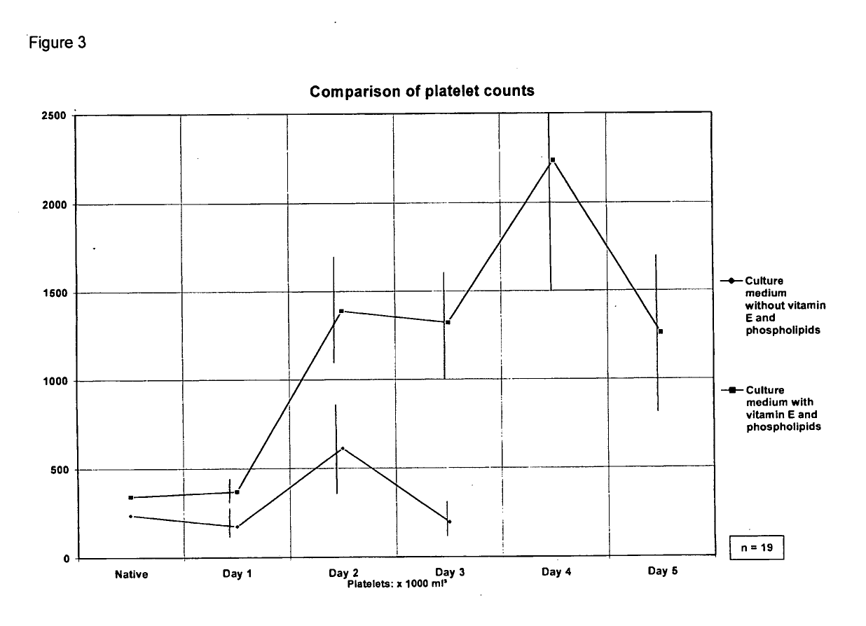

Platelets (thrombocytes), being a particularly sensitive substrate for proliferation-stimulating measures, increase from approx. 600,000 on day 2 of culturing in a culture medium not containing the supplements specified above to approx. 2.3 million on day 4 of culturing in a culture medium containing the supplements specified above (see FIG. 3).

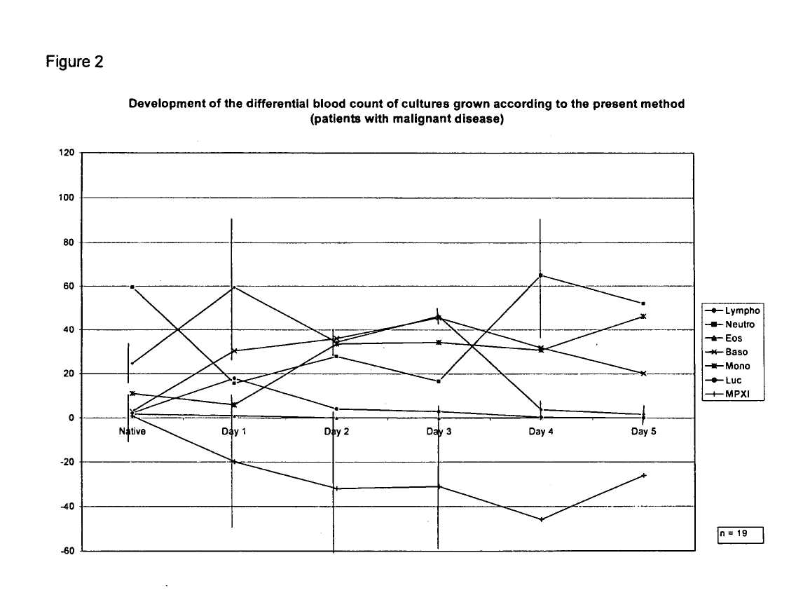

As a complete surprise, it turned out that not only the lifetime of a culture of this type and therefore the cell yield can be increased significantly, but also an unusual range of myeloic defense cells with an extraordinarily high fraction of macrophages is generated without prior selection such as by zone centrifugation. As is evident from the statistics and diagram above, approx. 30 to 50% monocytes and approx. 35 to 45% basophilic cells (see FIG. 2) are generated on days 3 to 5 of a culture of this type. All cell counts were determined by flow cytometry using an Advia 120 device made by Bayer Technics.

As is evident from the profile of the differential blood count under the culture conditions described above, the proliferation of the myeloic line increases on days 4 and 5 while the lymphocyte count decreases strongly concurrently. If the culture is boostered at this time by further addition of a vitamin E-proteoglycan mixture, the number of leukocytes, with particular emphasis on monocytes and basophils, increases to a level that exceeds previous experience in hematology. A cell density of up to several hundred-thousand leukocytes per cubic milliter is then attained.

As very well-equipped effector cells, basophilic leukocytes are, in a way, the end-product of cellular myeloic defense. The very large fraction of platelets, which are, to some extent, present in a culture of this type as giant platelets, also is evidence indicating potentiation of the antigen-antibody recognition process and also elevated macrophage activity which has also been conceded to these cells amongst other properties (see FIG. 3). In order to force this process, phospholipids from soybean as a fraction of the mixture are sufficient (see Line 40-44). This is surprising, on the one hand, because the quantitative distribution of soy phospholipids corresponds only incompletely to the structural distribution of the phospholipids of human cells or to the phospholipids of, e.g., bacterial walls, in particular of gram-negative bacteria, which are conceded to clearly have antigenic character with induction of, e.g., tumor necrosis factor. Moreover, soy phospholipids are advantageous in that they are virtually non-toxic if given via the parenteral route and can therefore even be administered intravenously in doses on the order of grams, which, e.g., is not true of MPL (monophosphoryl-lipid A, derivative of the lipopolysaccharide of a Salmonella species) which is used as an immunoadjuvant in hyposensitization. It can therefore only be administered in doses of up to 50 mg.

Comparing a standard culture to a phospholipid-stimulated whole-blood culture it is apparent that the lymphocytes decrease in favour of the myeloic line, the percentage of basophilic granulocytes is approximately twice as high, monocytes even increase 4- to 5-fold, and platelets increase extremely by comparison. (see FIGS. 2 and 3)

Upon activation, macrophages develop agglutinins and use them to adhere to the glass wall of the reaction vessel. Usually, activated macrophages are detached from the glass wall using trypsin solution. According to the invention, this is effected better using Haes solution (hydroxyethyl-starch), since this process proceeds much more gentle and leads to a higher cell survival rate.

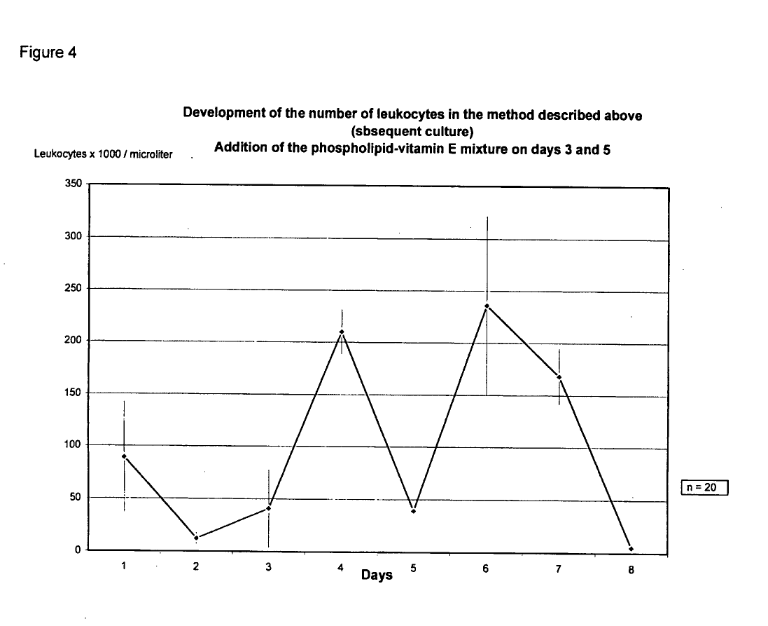

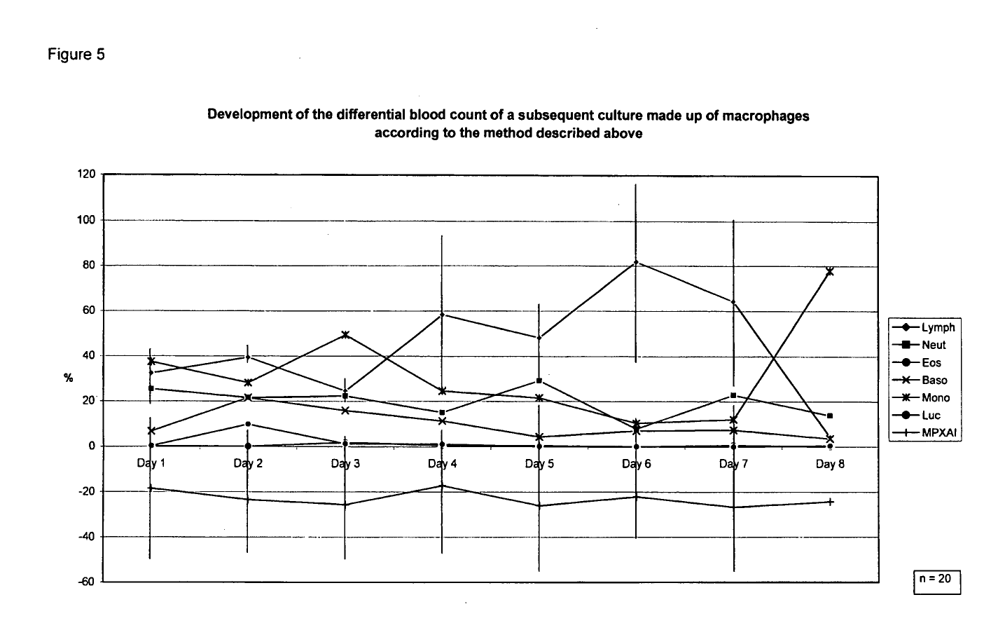

If a secondary culture of this type is stimulated according to the culture method described above, approximately 250,000 leukocytes per microliter are generated (FIG. 4) with an extraordinarily high fraction of monocytes on day 8 (FIG. 5).

The culture medium basically is made up by the ingredients described above in paragraphs [0004] to [0006]. As serum supplement serves the donor plasma that was treated with ozone at a concentration of 50-70 μg at twice to 4-fold the volume fraction (plasma:O2-O3 mixture equals [1:2 (4)]. The advantage of a culture medium processed as described is the tolerability of the medium for the autologous donor cells, the function of the ozonized plasma as O2 donor (up to more than 5.6×105 bar!), and the lack of autogenic pathogens due to ozonization. Moreover, it is the ideal substitute for bovine culture serum, which is commonly used in cell cultures since it is guaranteed to be free of prions.

The particularly high leukocyte yield on days 4 and 6 in a culture of this type is caused by the addition of the proliferation stimulants described above on days 3 and 5 (FIG. 4).

The monocytes of a secondary culture of this type can be used to obtain, in known manner according to the prior art, dendritic cells which have been shown in pilot studies throughout the world to be relatively effective against malignant diseases.

If the process of starting a new culture via activated macrophages that adhere to the wall of the reaction vessel, as described above, is repeated, the fraction of monocytes can be increased even further. Accordingly, the advantage of the method is not only to obtain monocyte-derived dendritic cells from this cell population, but also to offer the partners in antigen recognition and utilization, i.e. lymphocytes, simultaneously (see FIG. 5).

The control of the lymphocyte subpopulations showed a T-cell content of up to 30,000/mm³, which allows one to conclude that the phospholipid-vitamin E-proteoglycan mixture has a certain “antigen-booster effect”.

Due to its autologous origin, a preparation of this type that is obtained from an autologous blood preparation can be administered by the enteral and the parenteral routes with virtually no adverse effects. Despite the qualitative and quantitative changes of the autologous blood components, up to several milliliters of a preparation of this type, suitably diluted, can be administered intravenously.

Due to its triggering effects on the bone marrow, a medicinal agent according to the innovation described above can also be used with good prospects of success in leukopenia, thrombocytopenia, and cellular immune defects, in particular in those requiring elevated macrophage activity.

The extraordinary richness in monocytes and their special derivatives, the dendritic cells, in cultures of this type is an ideal prerequisite for the presentation of specific antigens. Accordingly, supplying antigens, for example attenuated viruses, polysaccharides or (ultra) centrifugation products of bacteria or fungi or cell fractions from malignant cells of autologous or homologous origin, to a monocyte-enriched autologous blood culture of this type, an enormous increase of the body's inherent specific defense is inevitable, depending on the selected antigen. Antigens of this type can suitably be obtained from bacterial cultures originating from autologous blood, urine or stool. After lyophilization and testing for reproductive capacity and sterility, pathogens of this type can be added as a stimulant to an autologous blood culture such that, after the end of culturing, a blood product is available as vaccine that can be used with very good prospects of success, for example, in cases of therapy-resistant infections. Especially autoimmune processes elicited by so-called focal infections respond very well to preparations of this type.

If the antigens described above are added to the culture either alone or in combination by adding the phospholipid/vitamin E mixture, a cell proliferation potentiation effect occurs that leads to extreme cell counts of up to 900,000 leukocytes/microliter.

If one adds to a whole-blood culture of this type half a milliliter of a standardized lysate of Diplococcus pneumoniae, Haemophilus influenzae, Streptococcus pyogenes and faecalis, Staphylococcus aureus, Klebsiella pneumoniae, Neisseria catarrhalis, flava and perflava, Gaffkya tetragena, and Moraxella, equivalent to a total number of 7.5 billion pathogens with an endotoxin content in excess of 10,000 units (measured using the Limulus test), their endotoxins can no longer be detected after completion of culturing, which is a surprising and clear indication of the unusually elevated macrophage activity that can be achieved with a whole-blood culture of this type.

Another individual-specific immune reaction can be attained by supplying these autologous blood cultures with allergens such as can be determined in clinical tests, for example Rast test, or pertinent laboratory tests. Usually, allergen mixtures of this type are used in the form of hyposensitization to develop a tolerance in the human organism. This therapy, whose efficacy has been documented in numerous controlled studies, has two decisive disadvantages though: firstly, it must be continued for years and, secondly, it is associated with a potential risk of shock with occasionally fatal outcome due to the direct contact of the organism and the allergen.

Because of its richness in immune-competent cells, an autologous blood culture of the type described above is virtually predestined for the production of specific immunoglobulins G, which are responsible for the development of tolerance as far as is currently known. A preparation of this type can therefore be used with good prospects of success without the disadvantages of hyposensitization described above.

The therapeutic use of bacterial antigens, e.g. in the form of killed bacteria, against malignant diseases has been known since the start of the last century. Also known is the use of BCG strains in various forms of malignant diseases, including in cultures of dendritic cells.

Numerous pilot studies report a different response of preparations of this type of origin. The direct application of bacterial suspensions in patients is obviously not without risk and per se associated with considerable side effects.

Likewise, the application of autologous blood cultures that are confronted with live bacteria is associated with considerable risks. However, if one rather uses exactly defined viral components, such as are, for example, used in vaccines (agglutinins and muraminidase), or lyophilization and ultracentrifugation products of bacteria or fungi, e.g. nuclear components or ribosomes, these risks do not exist. Preparations of this type are also offered on the market as finished pharmaceuticals.

Whereas the currently common allergy testing methods afford a reasonably certain antigen pattern for hyposensitization, the same cannot be realized, or only with inordinate laboratory efforts, e.g. by means of serological samples, for stimulation of cultures of this type in the case of bacterial or viral pathogens.

The method described above, though, offers a relatively simple opportunity for testing the sensitivity to antigens. The oxygen excess described above, in particular in the form of activated oxygen species, increases the sensitivity of white blood cells to antigens and thus changes their size, their number, and the amount of stainable biomaterial inside the cell. These parameters are easy to check in the individual measuring channels of modern automatic devices (so-called cell counters) for cell differentiation of the white blood count. Accordingly, if a culture sample of this type is confronted with the respective antigen, the loading of neutrophils with stainable biomaterial (Neut X, the cellular density of the neutrophils) as well as the substance of the cell nucleus (NEUT Y), and the volume of “shrunk” or “swollen” leukocytes (IMI DC) vary (see Operating Instructions of the cell counters made by Sysmex or Bayer Technics).

Monocytes, as antigen-recognizing cells, usually decrease, but their number may as well increase in particular cases and with a corresponding duration of culturing. Platelets as a particularly sensitive substrate react to the toxicity of the added antigen by decreasing in number, while the erythrocyte volume increases in line with the degree of toxicity of the antigen.

The procedure in this case is such that 50 μl antigen solution at a concentration that was determined empirically are added to a series of culture samples of the type described above, total quantity of heparin whole-blood (500 μl) or as blood with citrate added was treated with an O2-O3 mixture of 5-15 μg O3 concentration (10 ml gas per 20 ml of blood), and incubated for 6 to 12 hours in an incubating cabinet, e.g. at 37° C., or at room temperature. Subsequently, these mini-cultures are counted using a modern automatic cell counter device. This method is, except for the addition of the toxin to the culture samples, very easy and fast, and due to the plurality of determinations using a modern automatic cell counter very exact. The differences between the untreated culture and the antigen-supplied culture are determined and plotted in a graphic profile (see FIG. 6a: Neut X, FIG. 6b: Neut Y, and FIG. 6c: IMI DC). In FIG. 6c, the antigen-related shrinking and expansion processes are detected by the IMI DC channel.

These basic values are then used to determine “sensitivity factor 1” (who represents Micro- and Macrophaging activity) according to the following formula:

$$S F 1 = \frac{Δ Neut X \% \times Δ Neut Y \% \times Δ IMI DC \%}{Δ Mono \%} ( see Figure 6 d ) , \\ wherein {}_{}^{″}{Mono}{}_{}^{``} means absolute number of monocytes \text{:}$$

$$Δ Mono \% = \frac{( n Mono blank - n Mono contaminated )}{n Mono blank}$$

and “Neutr” means neutrophilic granulocytes; “Lympho” means lymphocytes; Neut X, Neut Y, and IMI DC mean the numbers determined via the corresponding measuring channels of the automatic counter device. The sensitivity factors thus determined are plotted in a graphic profile from which a selection of specific antigens can later be determined easily from the extent of the peaks of stimulation (see FIGS. 6a to 6e), which statistically reflect the results thus determined.

Antigens are known to differ in their affinity for specific cells. This is taken into account in further sensitivity factors 3 and 4 (see FIGS. 6e and 6f), whereby sensitivity factor 3 is calculated according to the formula

$$S F 3 = \frac{Δ Neut X \% \times Δ Neut Y \% \times Δ IMI DC \%}{Δ n Neutr \% \times Δ n Monocytes \% \times Δ n PLT \% \times Δ n Lympho \%}$$

In this formula, “n PLT”, “n Neutr.”, “n Lymph.”, and “n Mono” represent the absolute number of platelets, neutrophilic granulocytes, lymphocytes, and monocytes, respectively.

Sensitivity factor 4 is calculated according to the formula:

$$S F 4 = \frac{( Δ Neut X \% + Δ Neut Y \% + Δ IMI DC \% )}{( Δ n PLT \% + Δ n Neutr \% + Δ n Lympho \% )} \times Δ n Mono \%$$

Having monocytes in its numerator, sensitivity factor 4 represents mainly the antigen recognition of cellular immunity, whereas SF3 conveys the most comprehensive reflection of the influence of the antigens on the amount of leukocytes, platelets.

The optimization of the significance of the test by weighting individual groups of immunocompetent cells can be done not only by mathematical means, but also, in a simple manner, by separation of individual groups of cell.

This would be feasible, on the one hand, by separation in a centrifuge, on the other hand, in a relative simple manner, by storage. If a test culture of this type is stored for few days at refrigerator temperature or for a correspondingly shorter period of time at room temperature, free nuclear components are generated in the culture—and can be measured by the cell counter—which are identified by the cell counter to be granulocytes or components thereof.

Usually, this process has reached sufficient intensity at room temperature after as little as 6 to 48 hours for lymphocytes and monocytes to occur as the main reaction partners for the toxins in this test procedure.

Accordingly, control of the contaminated test sample maximally 6 hours after inoculation results in a useful statement taking into consideration the granulocytes, and a clearly differentiated statement with a clear emphasis on lymphocytes and monocytes after two days of storage.

The results of this test method after several days of storage at refrigerator temperature (2° C.-8° C.) can therefore be implemented in the culturing of an autologous blood culture, i.e. cold storage for 2-6 days of the stored blood prior to culturing inevitably leads to lymphocytes and monocytes prevailing in the final product, and therefore leads to selection and optimization of the therapeutic range.

It is evident from FIGS. 6a to 6f that the results in the population of neurodermatitis patients is virtually identical to the clinical experience made thus far.

Selecting stimulation factors according to the method according to the invention and supplying antigens selected in this way to autologous blood cultures has proven useful not only for diagnostic purposes, but also has led to remarkable clinical successes. Two previously unknown phenomena became apparent during the control of cultures stimulated as described:

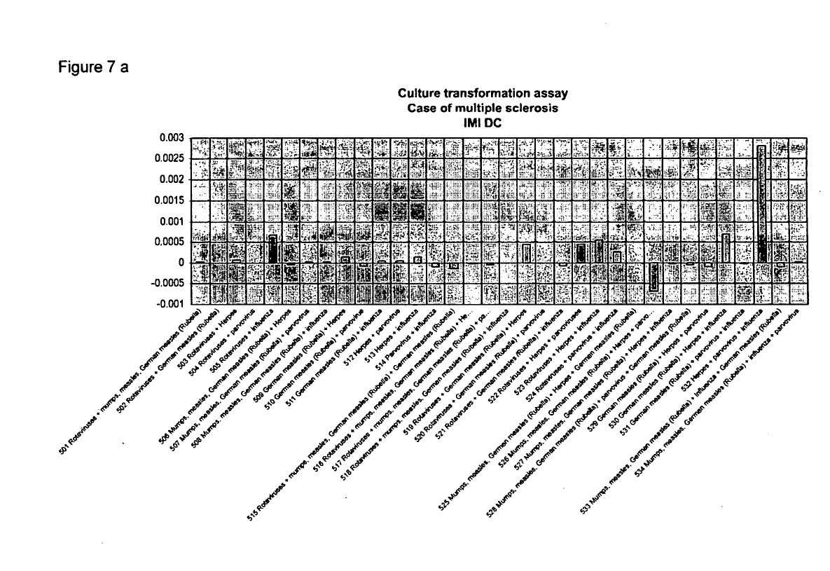

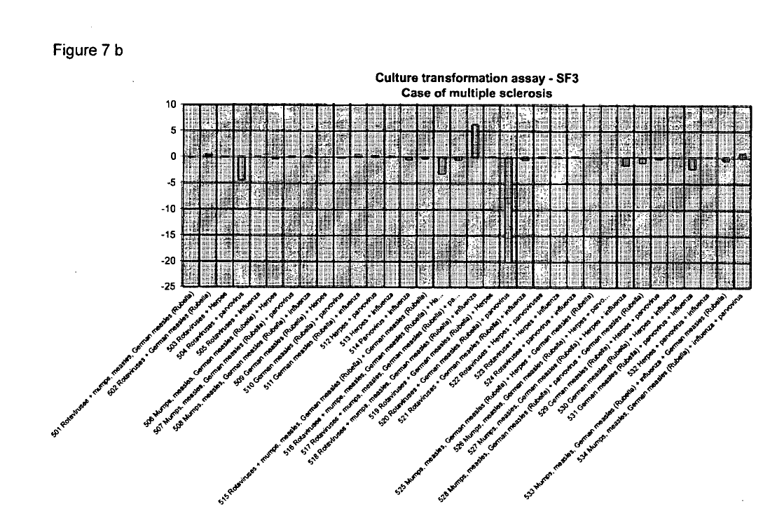

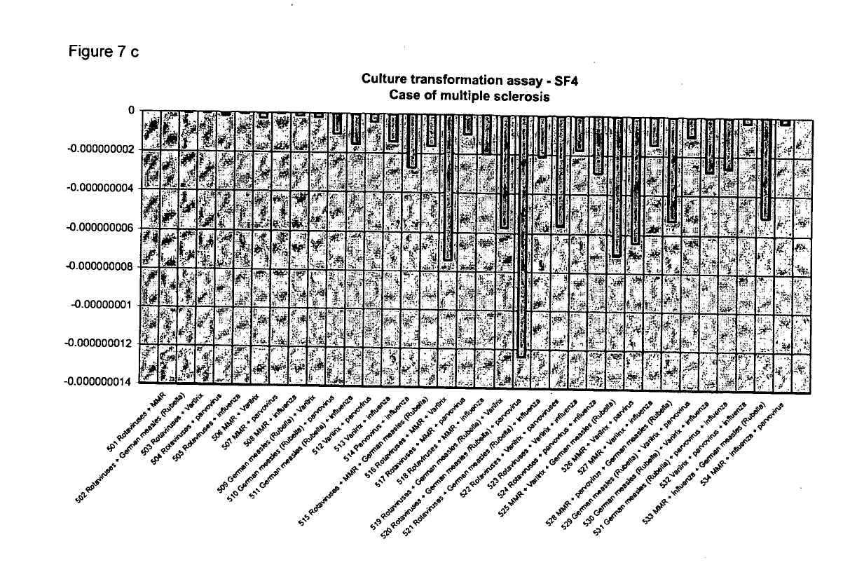

especially at the level of the IMI DC (toxicity effects), combination of certain antigens afforded a remarkably strong toxic effect in some indications, i.e. the toxic effect of selected pathogens, used in combination, showed a potentiating toxic effect on the immunocompetent cell [see FIGS. 7a, 7b, and 7c: Effect of the combination of herpes viruses, rubella viruses (number 509, see FIG. 7c), whereas a combination of herpes viruses, rubella and parvoviruses (number 529, see FIG. 7b) was associated with abolishment of the toxic effect of parvoviruses (competition for occupancy of the same receptors)].

Accordingly, there are combinations of pathogens that have a subtractive effect on the toxic or even just the immune system-modulating effect elicited by a virus. The test thus enables a person skilled in the art to either exert a potentiating effect on the immune system or to reduce or even abolish the damaging effect of a virus by combining it with a second or more virus(es) (e.g. by receptor occupancy) by selecting specific pathogen combinations.

The toxic effect of individual antigens or selected antigen combinations can go so far that a culture of this type can be destroyed within a few hours. A process that even enables diagnostic utility at high reliability, in which, for example, the destruction potential of the respective antigen is measured per unit of time.

As is evident from the further figures, cell suspensions varying in composition can be obtained as a function of the culture time depending on the addition of stimulation material.

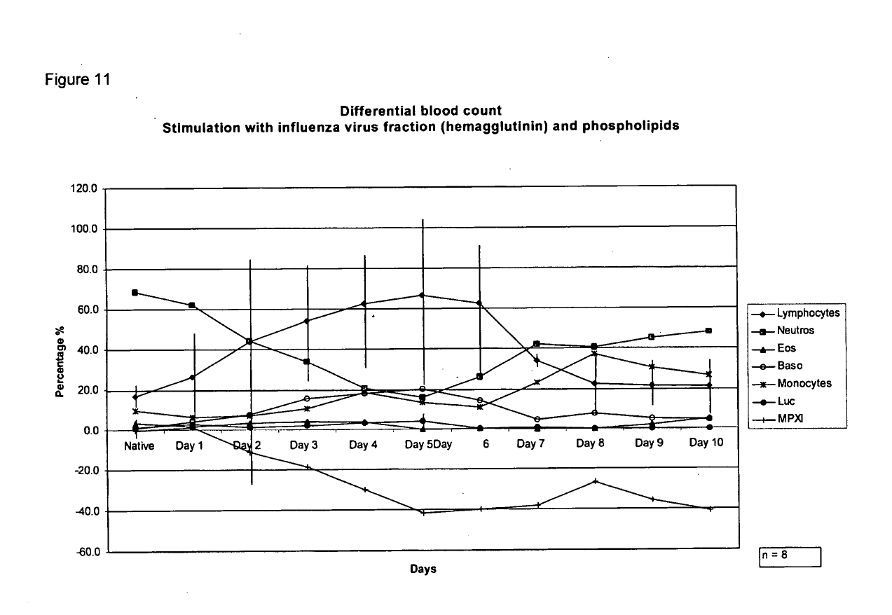

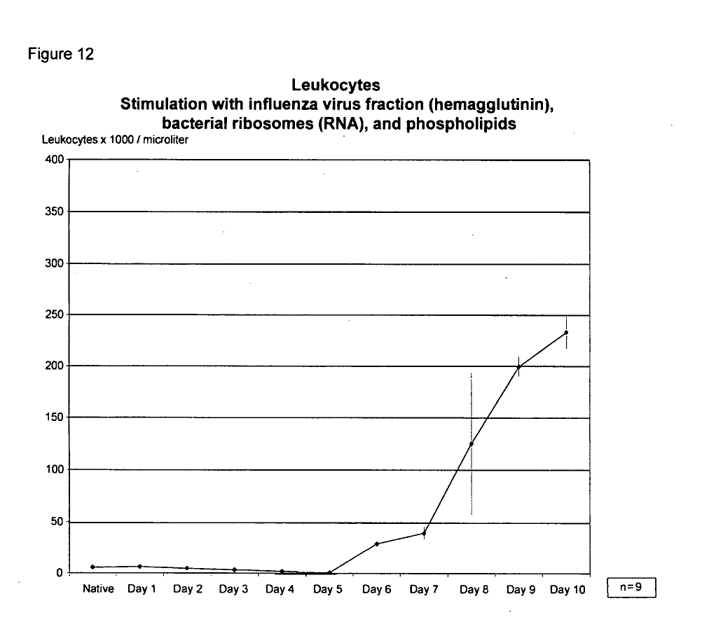

The subsequent culture of an autologous blood culture that was stimulated according to the method according to the invention according to FIG. 11 and FIG. 12 is very rich in lymphocytes and thus lends itself to being used where there are symptoms of a lack of specific antibodies.

As is evident from the profiles of the various subpopulations of the myeloic lines, leukocytes, lymphocytes, and other cell lines reach their peaks at different points in time depending on the selection and combination of antigens, which suggests to carry out the stimulations described above at an optimized lead time—depending on the clinical indication.

If an optimized antigen combination is detected based on the culture transformation assay, e.g. in the case of malignant diseases, it is not uncommon to detect a 40% to 90% increase in eosinophilic granulocytes in the culture, which is usually a sign of a successful, virtually side effect-free treatment of cancer diseases.

The use of monocyte-enriched cultures that have been stimulated with bacterial or viral fractions in malignant diseases leads to a very rapid decline of the tumor markers in just a few days in many of the patients thus treated, e.g. in the case of pancreatic, ovarian, colon, and prostate cancer.

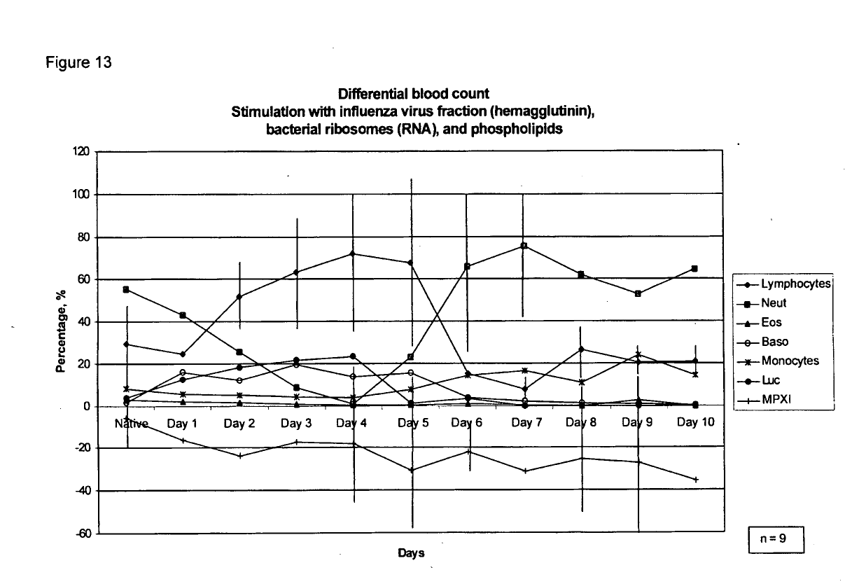

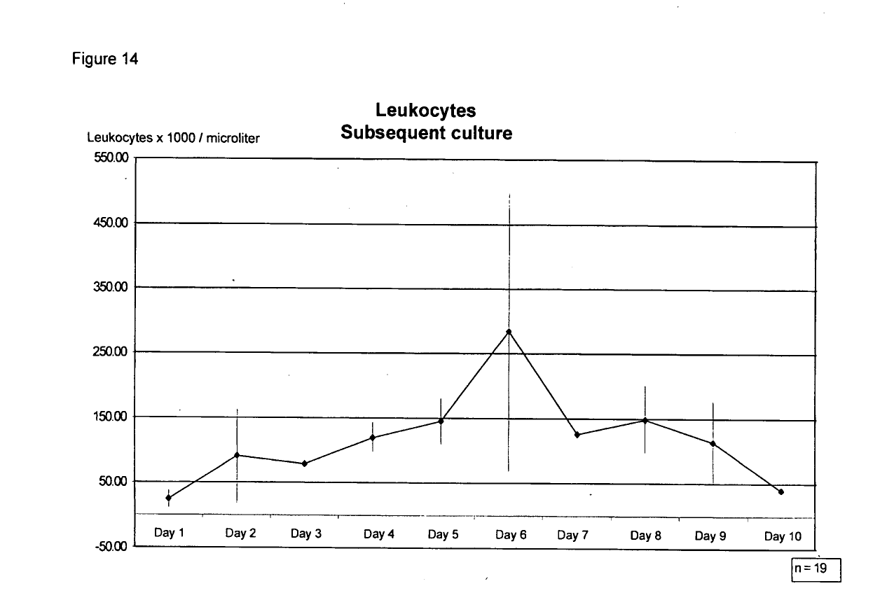

As is shown by FIGS. 13 and 14 and is evident from the results of the culture transformation assay (7b and 7c), the addition of bacterial or viral components effects a transient decrease in the total number of leukocytes and of individual cell lines of the myeloic line.

Accordingly, if a culture of this type is contaminated, for example, with a plurivalent vaccine, the number of leukocytes may decrease to just a few hundred cells per microliter, which inevitably necessitates a long time for the culture to recover until a therapeutically reasonable cell concentration range is reached again. The innovation therefore proposes to bridge this shortcoming in indications such as, for example, a polytopic immune deficiency, through multiple inoculation of the culture on multiple days, resulting from the innovation, by splitting the original culture into several individual cultures, stimulating them separately, and reintegrating them thereafter.

This allows an extraordinarily potent “hyperimmune” blood preparation to be produced that is not only tolerated very well, but can also be called pluripotent because of the multiple stimulation.

A subsequent culture following the steps described above can subsequently be cultured without further stimulation for many weeks (current observation time is up to one quarter of a year) and reaches cell counts of up to 200,000 per μl. This pool can then be “harvested” and again followed by renewed stimulation of cell growth by the culture medium described above.

Just as it is feasible to produce an autologous, plurivalent “hyperimmune” blood preparation by this means, it is also feasible to produce a monovalent or plurivalent pool preparation from multiple donors of the same blood type in this manner which can then be injected into recipients of the same blood type.

Particularly good tolerability is to be expected in the case of preparations which can be obtained according to the prior art from the cell-free supernatant or centrifugation product of cultures of this type.

In summary, an autologous whole-blood culture is proposed that is nurtured and stimulated by a culture medium which contains, aside from the classical components of electrolytes and amino acids, a high fraction of phospholipids, predominantly of plant origin, vitamin E, and low-molecular proteoglycans. The protein base of the culture medium consists of autologous ozonized plasma. The use of a culture medium of this type leads to forced cell growth. The excess of activated macrophages observed in the process is detached from the wall of the reaction vessel by hydroxyethyl-starch solution and used for further subsequent culturing.

The final product of culture processes of this type is characterized by an extremely high fraction of monocytes, basophilic granulocytes, and platelets, and thus lends itself as a medicinal agent for cellular immune defects.

Moreover, stimulation of these cultures with a mixture of phospholipids and antigens of viral, bacterial, mycotic or cancerogenic origin, but also with allergens, allows for its use for specific defense in this context or for tolerance development in the organism. A method for selection of specific antigens as stimulants for these cultures that is based on the change of hematological parameters of the whole-blood serving as culture material is proposed.

The selection method allows for combining pathogens that potentiate each others' immune system-modulating effect or mutually inhibit each others' damaging effect on the organism.

A further useful diagnostic method is the measurement of leukocyte degranulation. According to the art this is done by microscopic procedures, a relatively laborious method. An improved process includes via several staining techniques also the leukocyte subpopulations. The method described above using a cell counter is useful for determining the cell degranulation and also the induction of RNA and the toxic effects on thrombocytes.

For determining the cell degranulation of leukocytes the factor Neut X is equated with the factor IMI DC according to the formula

$$Leukocyte degranulation = \frac{Neut X blank + \text{/} - Neut X K}{Neut X blank} \times \frac{IMI DC blank + \text{/} - IMI DC K}{IMI DC blank}$$

with Neut blank being the granularity of the Neut X channel of the non contaminated blood count, Neut X K being the granularity of the Neut X channel of the contaminated blood count, IMI DC being the volume of the non contaminated cells found in the IMI DC channel and IMI DC K the volume of the contaminated cells.

Since contaminated cells may shrink in their volume during degranulation or depending on the intensity of the toxic effect may swell in the same time and increase the granularity the formula provides a surprisingly exact picture of the toxic effect of an antigen onto cell degranulation.

The substance of the cell nucleus of the myeloic line consists of DNA and RNA, whereby the RNA is the messenger substance for multiple functional structures of the cytoplasm. If one multiplies the change in granularity with the change of the cell nucleus according to the formula

$$RNA induction = \frac{Neut Y blank + \text{/} - Neut Y K}{Neut Y blank} \times \frac{Neut X blank + \text{/} - Neut X K}{Neut X blank}$$

the RNA induction that the antigen causes within the cells can be deduced based on the loss of RNA in the nucleus and the induction of the granula in the cytoplasm. This method provides a surprisingly instructive conclusion e.g. on the influence of viruses on the cell due to the specific affinity of viruses to the cell nucleus.

Thrombocytes have not been taken into account during diagnostic screening of toxic effects on the blood count so far. However, thrombocytes change their size under the influence of multiple toxins, i.e. they grow in a manner that can be determined exactly with the cell counter. Concurrently the mean number of thrombocytes per mm3 decreases depending on the toxic effect of the antigen. A complete overview on the toxic effect of different antigens on the thrombocytes is obtained according to the formula

$$P A F = \frac{Thromb N blank - Thromb N K}{Throm N blank} \times \frac{Thromb V blank - Thromb V K}{Thromb V blank}$$

wherein Thromb N blank is the number of thrombocytes in the non contaminated condition and Throm N K is the number of thrombocytes in the contaminated condition. Thromb V blank is the volume of the thrombocytes without contamination, Thromb V K the volume of the thrombocytes under the influences of the toxin. The result of such a calculation corresponds substantially to the platelet activating factor (PAF). Validity of this conclusion is proven by the significant increase above the statistical mean value of the effect of chlamydiae on the thrombocytic system during immuno vasculitides and other vascular diseases according to this calculation method. Chlamydiae have repeatedly been isolated from substance obtained in arterial surgery characterized by plaque-like precipitates on the intima or by vascular obliteration. Further indication in this direction is the frequent consistency of the PAF determined according to this formula with the leukocyte degranulation or the overall influence on the myeloic line as described above. Induction of the PAF by a toxin induces a whole range of leukotrienes, which in turn are detected by leukocyte degranulation and the toxic effect on the myeloic blood count.

Determination of the toxic effects according to the methods described above allow an exact choice of toxines that can later also be used to stimulate autologous blood cultures. For example, papilloma viruses, which are known to be the cause of cervix carcinomas and are also relatively often found with bronchial carcinoma can be detected with this method as co-factores of such diseases. The same applies to chlamydiae in the context of arterial diseases as mentioned already, for several types of funghi, especially spur-bearing, in the context of neurodermatitis, rubella viruses in the context of neurodermatitis and psoriasis, streptococcus bacteria of pulmonal and enteral origin in the context of primary chronic polyarthritis or arthritis psoriatrica, but also rubeola viruses as co-factors of the clear cell associated bronchial carcinoma, as has been shown with other methods already. Thus, the invention also provides a method for identifying toxins and co-factors as cause of diseases using a cell counter wherein the cell degranulation of the myeloic line, the RNA induction and the platelet activating factor are determined through morphological controls and counting. The toxic factors identified in this way can then be used for stimulating autologous cell cultures in therapy.

Unless specified otherwise or where evident from the context, all specifications made in units of % in the present invention refer to the total weight of the mixture.

1-45. (canceled)

46. A whole-blood culture containing specific immunocompetent cells that are activated against tumor cells, viruses, bacteria and/or allergens, wherein the whole-blood culture comprises of whole-blood and a culture medium at a ratio of 3:1 to 4:1, wherein the culture medium
has an oxygen excess of at least 100% or more, and
contains a water-soluble emulsification product comprising a mixture of phospholipids, vitamin E, and low-molecular proteoglycans with a molecular weight of 1,200 to 12,000 Dalton, and
wherein dead or living tumor cells or fragments thereof and/or viral and/or bacterial antigens and/or allergens have been added to the whole-blood culture to serve as antigen in the specific recognition process for production of the activated killer cells.

47. A whole-blood culture according to claim 46, wherein the proteoglycans are pentosan polysulfate.

48. A whole-blood culture according to claim 46, wherein the culture medium contains folic acid.

49. A whole-blood culture according to claim 46, wherein the mixture of phospholipids, vitamin E, and low-molecular proteoglycans accounts for a weight fraction of at least 10% of the weight of the culture medium.

50. A whole-blood culture according to claim 46, wherein at least 80% of the phospholipids are phosphatidylcholine.

51. A whole-blood culture according to claim 46, wherein the whole-blood culture has been cultured for three to five days and comprises of
a monocyte fraction of 30 to 60 wt-%;
a basophilic granulocyte fraction of >30 wt-%; and
a platelet fraction of >1 million per cubic-millimeter.

52. A whole-blood culture according to claim 46, wherein it is composed of multiple autologous individual cultures that have been stimulated with different antigens.

53. A whole-blood culture according to claim 46, wherein in the whole-blood culture, the culture medium has a serum base that comprises of autologous ozonized donor plasma.

54. A method for the producing a whole-blood culture containing specific killer cells that are activated against tumor cells, viruses, bacteria and/or allergens, comprising the steps:
culturing whole-blood and a culture medium made up of a mixture of phospholipids, vitamin E, and low-molecular proteoglycans with a molecular weight of 1,200 to 12,000 Dalton at a whole-blood-to-culture medium ratio of 3:1 to 4:1;
addition of dead tumor cells or fragments thereof and/or viral antigens and/or bacterial antigens and/or allergens as antigen in the specific recognition process (stimulation) of the developing defense cells;
supplying an oxygen excess of at least 100% to the culture medium.

55. Method according to claim 54, comprising the steps of repeated boostering of the whole-blood culture with a mixture of vitamin E, phospholipids and/or proteoglycans.

56. Method according to claim 54, comprising the step of detaching adhesive cells of the whole-blood culture from a wall of a culture vessel, in which the whole-blood culture is cultured, by adding a Haes solution, in particular a hydroxyethyl-starch.

57. Method according to claim 54, comprising the step of stimulating the whole-blood culture:
with one of antigens of attenuated life or dead viruses, lyophilization products of bacteria or fungi or polysaccharides and other fractions of bacterial or mycotic origin, fractions from malignant cells or allergens of animal or plant origin or combinations thereof,
or with a mixture comprising virus fractions, bacterial and/or mycotic components and phospholipids, whereby the fraction of phospholipids is at least 20%.

58. A method according to 54, comprising the step of obtaining an autologous or homologous cell-free serum from a supernatant or centrifugation product of the whole-blood culture.

59. A method for selecting a stimulant that is well-suited for use for stimulation of a whole-blood culture according to claim 46, comprising the steps:
contaminating the blood of a donor with an antigen;
measuring the changes of the cellular phase of the blood of a donor by means of a measuring series of pre-cultures;
determining the specific antigens that are well-suited for stimulation by comparing measuring results regarding the resulting changes of the cellular phase that have been obtained for at least two different antigens used for contamination.

60. A method according to claim 59, comprising the step of exposing the pre-cultures of the blood of the donor to an ozone-oxygen mixture prior to the step of contaminating with antigen.

61. A method according to claim 59, comprising the step of taking into consideration morphological and numerical changes, in particular numerical changes of monocytes, platelets, lymphocytes or their subpopulations or of erythrocytes, of the differential blood count of an antigen-contaminated culture as compared to at least one untreated culture as hematological criteria.

62. A method according to claim 61, wherein
a loading of neutrophils with stainable biomaterial (Neut X);
a substance of the cell nucleus (Neut Y); and
a volume of shrunk or expanded leukocytes (IMI DC) or a combination of the criteria mentioned above serve(s) as hematological criteria.

63. A method according to claim 62, comprising the step of determining a damaging effect of the antigen on a cellular blood count according to the formula
$$S F 1 = \frac{Δ Neut X \% \times Δ Neut Y \% \times Δ IMI DC \%}{Δ n Mono \%}$$
wherein SF1 expresses the summarized toxicity to the inside of the cell and wherein “Mono” means the absolute number of monocytes:
$$Δ Mono \% = \frac{( n Mono blank - n Mono contaminated )}{n Mono blank} .$$

64. A method according to claim 62, comprising the step of determining an effect of the antigen on a cellular phase of an immune system morphologically and numerically according to the formula
$$S F 3 = \frac{Δ Neut X \% \times Δ Neut Y \% \times Δ IMI DC \%}{Δ n Neutr \% \times Δ n Monocytes \% \times Δ n PLT \% \times Δ n Lympho \%}$$
wherein SF3 summarizes the toxicity to the inside of the cell and Neutr means neutrophils, Lympho means lymphocytes, and Neut X, Neut Y, and IMI DC represent the values that are determined via the corresponding measuring channels of an automatic cell counter.

65. A method according to claim 62, comprising the step of recognizing the antigen according to the formula
$$S F 4 = \frac{( Δ Neut X \% + Δ Neut Y \% + Δ IMI DC \% )}{( Δ n PLT \% + Δ n Neutr \% + Δ n Lympho \% )} \times Δ n Mono \%$$
wherein SF4 mainly captures the cellular immunity.

66. A method according to claim 59, comprising the step of selecting the antigen or antigen combination taking into consideration an effect that takes into account the toxicity of the antigen or antigen combination on cells per unit of time.

67. Stimulant suitable for stimulation of a whole-blood culture according to claim 46, wherein in that the stimulant comprises bacterial components that are mono- or mixed cultures obtained from faeces or urine of the organism to be treated.

68. A medicinal agent for immunostimulation or treatment of immune defect or malignant diseases, which comprises a whole-blood culture or a cell-free serum from a supernatant or centrifugation product of the whole-blood culture according to claim 46.

69. Method for immunostimulation or treatment of immune defects or malignant diseases comprising the steps
identification of a patient in need of treatment;
production of a whole-blood culture and selection of a stimulant for stimulation of the immunocompetent cells according to claim 59;
administration of the whole-blood culture or a cell-free serum from a supernatant or centrifugation product of the whole-blood culture.

70. A method according to claim 59, comprising the step of taking into consideration morphological and numerical changes, in particular numerical changes of the cell degranulation of the myeloic line, the RNA induction and the platelet activating factor of the differential blood count of an antigen-contaminated culture as compared to at least one untreated culture as hematological criteria.

71. A method according to claim 70, wherein the leukocyte degranulation is determined according to the formula
$$leukocyte degranulation = \frac{Neut X blank + \text{/} - Neut X K}{Neut X blank} \times \frac{IMI DC blank + \text{/} - IMI DC K}{IMI DC blank} .$$

72. A method according to claim 70, wherein the RNA induction is determined according to the formula
$$R N A Induction \frac{Neut Y blank + \text{/} - Neut Y K}{Neut Y blank} \times \frac{Neut X blank + \text{/} - Neut X K}{Neut X blank} .$$

73. A method according to claim 70, wherein a toxic effect of an antigen onto the platelet activating factor is determined according to the formula
$$P A F Induction = \frac{Thromb N blank - Thromb N K}{Thromb N blank} \times \frac{Thromb B blank + Thromb V K}{Thromb V blank} .$$

74. Method of stimulating autologous blood cultures wherein an antigen identified according to claim 70 is used.
